(19) United States
(12) Patent Application Publication
(10) Pub. No.: US 2023/0112497 A1
(43) Pub. Date: Apr. 13, 2023

(54) PROVISIONING SYSTEM FOR ENDOSCOPES

(71) Applicant: Olympus Winter & Ibe GmbH, Hamburg (DE)

(72) Inventors: Ralf Siegmund, Ahrensburg (DE); Daniel Zuewers, Hamburg (DE); Jan Niebuhr, Kiel (DE); Christoph Alexander Ahrens, Reinbek (DE); Sascha Jaskola, Hamburg (DE); Mathias Hueber, Hamburg (DE); Ralf Tessmann, Hamburg (DE); Stefan Schroeder, Hamburg (DE)

(73) Assignee: Olympus Winter & Ibe GmbH, Hamburg (DE)

(21) Appl. No.: 17/963,443

(22) Filed: Oct. 11, 2022

Related U.S. Application Data
(60) Provisional application No. 63/255,050, filed on Oct. 13, 2021.

(51) Int. Cl. G06F 3/048

(52) U.S. Cl. CPC G06F 3/048; G06F 3/0488

(57) ABSTRACT

A provisioning system for endoscopes in an application environment including one or more examination rooms and one or more reprocessing stations for endoscopes. The provisioning system including a processor including hardware. The processor being configured to: receive first information about scheduled procedures, receive second information about a status of one or more endoscopes, the second information including an indication of whether an endoscope is ready for use, currently in use, or currently being reprocessed, determine from the first information and the second information whether a ready-to-use endoscope is or will be available for each respective scheduled procedure, and control a display to display an indicator which indicates whether a ready-to-use endoscope is or will be available for the respective scheduled procedure.

### CROSS-REFERENCE TO RELATED APPLICATION

The present application is based upon and claims the benefit of priority from U.S. Provisional Application No. 63/255,050 filed on Oct. 13, 2021, the entire contents of which is incorporated herein by reference.

### BACKGROUND

### Field

The present disclosure relates to a provisioning system for endoscopes.

### Prior Art

Endoscopes have long been used in medicine to examine or treat cavities of a patient's body that are difficult to access. They are generally reusable and must undergo a complex reprocessing process after each use before they can be used to examine or treat another patient.

A reprocessing process, for gastroenterological endoscopes, usually includes manual precleaning, machine cleaning and disinfection, drying if necessary, and storage in a supply cabinet.

Offices or departments specialized in endoscopic procedures regularly comprise a large number of treatment rooms in which several procedures can be performed in parallel. For this purpose, a large number of endoscopes of different types are kept in stock, and several manual and mechanical reprocessing stations are provided for their reprocessing. Within the framework of occupancy planning, it is determined for the individual treatment rooms when which procedures are to be carried out with which endoscopes or endoscope types, and the patients to be treated in each case are appointed accordingly. In the occupancy planning, it can also be determined at which reprocessing stations the endoscopes are reprocessed after use.

In occupancy planning, some parameters must first be estimated. These include, for example, the duration of individual procedures and the duration of reprocessing of individual endoscopes before they can be used again. Deviations in the actual duration of a procedure or a reprocessing process can have significant consequences if, for example, an endoscope is not available in time to be subjected to machine cleaning and disinfection together with other endoscopes after manual pre-cleaning due to a longer procedure. In this case, either the start of the machine cleaning and disinfection must be delayed, as a result of which all the endoscopes concerned are not available for reuse until later, or the endoscope that is not available until later must be provided for a later am of the machine cleaning and disinfection, as a result of which the re-provision of the endoscope concerned is delayed even further.

A system for managing endoscopes is known from U.S. Pat. No. 8,768,721 B2, which determines the possible effects of a reprocessing capacity failure on occupancy planning and uses this to determine an additional requirement for endoscopes, which are then procured as items on loan or on purchase. However, it is not always possible to procure additional endoscopes at short notice.

However, it is difficult for a person responsible for occupancy planning to foresee the actual impact of delays in individual procedures or reprocessing processes on current occupancy planning.

Furthermore, the estimated values of the individual process durations available for occupancy planning allow only limited accuracy in planning.

### SUMMARY

Therefore, an object is to develop an improved provisioning system for endoscopes.

Such objective can be achieved by a provisioning system for endoscopes in an application environment comprising one or more examination rooms and one or more reprocessing stations for endoscopes, the provisioning system being configured to receive first information about scheduled procedures and to receive second information about a status of one or more endoscopes, the second information comprising an indication of whether an endoscope is ready for use, currently in use, or currently being reprocessed, and wherein the provisioning system is configured to determine from the first information and the second information whether a ready-to-use endoscope is or will be available for each of the scheduled procedures. The provisioning system can further comprise a user interface having a graphical user interface, wherein the graphical user interface can include a first presentation area in which data about ready-to-use endoscopes is displayed, a second presentation area in which data about scheduled procedures is displayed, and a third presentation area in which data about endoscopes undergoing reprocessing is displayed, wherein the data displayed in the second presentation area comprises an indicator which indicates whether a ready-to-use endoscope is or will be available for the respective procedure.

Receiving the second information may comprise receiving data that does not directly describe the state of an endoscope but allows conclusions to be drawn about it, and further evaluating this data to determine the state of the endoscope. Additionally, a location of an endoscope may also be determined.

By the appropriate embodiment of the provisioning system, information about the availability of the endoscopes can be offered in such a way that a user can intuitively grasp it and immediately recognize whether a required endoscope will be available in time for all planned procedures.

In a further embodiment of a provisioning system, the provisioning system may be configured, in a case in which an endoscope will not be available in time for a scheduled procedure, to determine possible modifications to ongoing or pending reprocessing processes by which a delay in the provision of the endoscope concerned can be reduced.

The provisioning system may be configured to determine the possible modifications upon request by a user. Further, the provisioning system may be configured to provide determined possible modifications in the form of a selection list.

In this way, in addition to the information as to whether an endoscope will be available for all procedures, a user of the provisioning system can be quickly and easily offered possible solutions in the event of problems with the provision, in order to reduce or even completely avoid influences of the delayed provision on the examination procedure.

The provisioning system may be configured to generate and send control commands to an endoscope reprocessing device upon selection of a determined modification by the user in order to cause the endoscope reprocessing device to perform a modified reprocessing process. Similarly, the provisioning system may be configured, upon selection of a determined modification by the user, to generate and transmit execution instructions for a modified reprocessing process in text form to a manual pre-cleaning station for display.

Depending on whether the preparation process or preparation step to be modified is a process or process step performed by machine or manually, the modification can thus be implemented easily and effectively.

The provision system may comprise a control memory in which rules are stored according to which process parameters of reprocessing processes can be modified without impairing the effectiveness of the reprocessing processes.

Performance curve fields may be stored in the control memory, which describe the effectiveness of a reprocessing step as a function of one or more process parameters.

By a corresponding control memory and, if applicable, performance curve fields stored therein, possible modifications of reprocessing processes can be determined effectively.

According to another aspect, such object can be achieved by a method for operating a provisioning system for endoscopes, comprising: receiving first information about scheduled procedures, receiving second information about a status of one or more endoscopes, the second information comprising an indication of whether an endoscope is ready for use, currently in use, or currently being reprocessed, and determining, from the first information and the second information, whether a ready-to-use endoscope is or will be available for each of the scheduled procedures, and displaying the first and second information in a graphical user interface. Therein, the graphical user interface can include a first presentation area in which data about ready-to-use endoscopes is displayed, a second presentation area in which data about scheduled procedures is displayed, and a third presentation area in which data about endoscopes undergoing reprocessing is displayed, wherein an indicator can displayed in the second presentation area, which indicates whether a ready-to-use endoscope is or will be available for the respective procedure.

With regard to possible further developments as well as the advantages and effects achievable thereby, express reference is made to what has been said above.

### BRIEF DESCRIPTION OF THE DRAWINGS

In the following, embodiments of a reprocessing system for endoscopes will be described in more detail with reference to some exemplary figures. In this regard, the illustration serves only to provide a better understanding of the invention without limiting it, in which:

FIG. 1 illustrates a provisioning system for endoscopes.

FIG. 2 illustrates a user interface.

FIG. 3 illustrates another user interface.

FIG. 4 illustrates a performance curve field.

### DETAILED DESCRIPTION

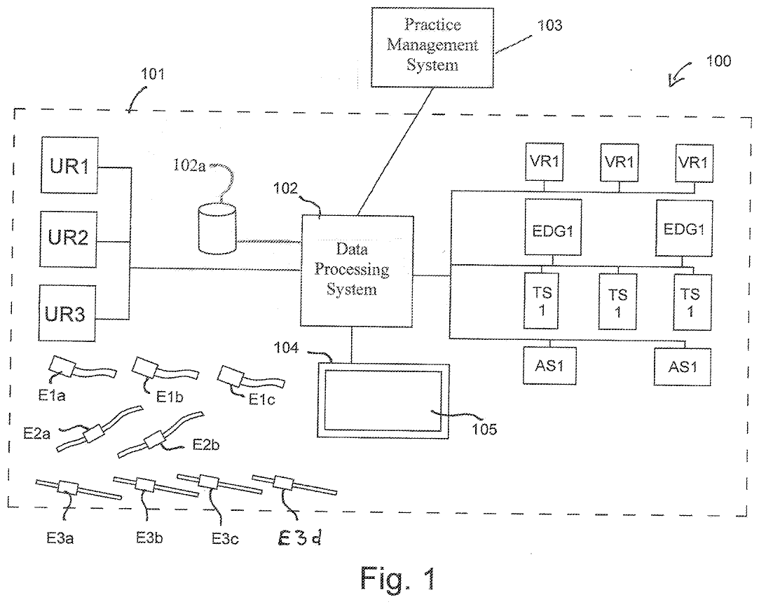

FIG. 1 shows an endoscope provisioning system 100 in an exemplary application environment 101, which may be an outpatient endoscopy office or an endoscopy department of a hospital.

The application environment includes a plurality of examination rooms UR1, UR2, UR3. The number of examination rooms may vary as desired. The term “examination mom” does not exclude that procedures with interventional parts such as biopsies or sclerotherapy are also performed in these rooms.

Furthermore, the application environment comprises a reprocessing station for the reprocessing of used endoscopes, which includes manual pre-cleaning stations VR1, VR2, VR3, endoscope reprocessing units EDG1, EDG2, drying cabinets TS1, TS2, TS3, and storage cabinets AS1, AS2. The numbers of the individual elements are again arbitrary and serve only as an example.

To perform endoscopic examinations in the examination rooms UR1, UR2, UR3, several endoscopes of different types are kept available. For example, endoscopes E1a, E1b, E1c of a first type E1, endoscopes E2a, E2b of a second type E2, and endoscopes E3a, E3b, E3c, E3d of a third type E3 are kept available. The endoscope types E1, E2, and E3 can be gastroscopes, bronchoscopes, and colonoscopes. Of course, endoscopes of other types may also be present.

The provisioning, system 100 includes a data processing system 102 configured to receive and process information related to scheduled and/or ongoing procedures and reprocessing processes, as well as the current location and condition of individual endoscopes, if applicable. The data processing system 102 can be embodied by software running on a processor, controller, CPU or regular or special purpose computer (e.g., medical-grade computer) and each may have an associated storage device (memory).

Information about scheduled procedures may be provided to the data processing system 102, for example, by a hospital or practice management system 103. The office management system 103 can be embodied by software running on a processor. controller, CPU or regular or special purpose computer (e.g., medical-grade computer) and each may have an associated storage device (memory). This may be provided via an interface, for example using the DICOM standard. Such information may include the type of procedure, the scheduled start of the procedure, and the examination room in which the procedure is to be performed.

Information about ongoing reprocessing procedures is usually provided by the endoscope reprocessing equipment.

To determine the location of individual endoscopes, sensors are provided in the examination rooms UR1, UR2, UR3, as well as at the reprocessing stations, which detect identification features of endoscopes located in the examination room or at the reprocessing station. Such sensors may include, for example, RFID readers that detect RFID tags individually assigned to the endoscopes, either automatically or manually controlled.

Information about the current status of individual endoscopes is largely derived from the above. For example, if an endoscope is in an examination room, the status may be assumed to be that the endoscope is in use. If, on the other hand, the endoscope is at a manual pre-cleaning station, the state may be assumed to be that the endoscope is currently being pre-cleaned manually. In this case, it may also be recorded how long the endoscope has been in the pre-cleaning, process.

The EDG1, EDG2 endoscope reprocessing devices usually additionally provide information about the type and progress of the selected reprocessing program, which also indicates the status of the endoscopes that are in the respective endoscope reprocessing device.

From the received data, the provisioning system 100 determines whether suitable endoscopes are available for each of the scheduled procedures. To do this, one or more compatible endoscope types with which the procedure can be performed are assigned to each planned procedure. At the same time, the number of endoscopes currently available of each endoscope type is determined. An endoscope is considered to be available if it has been completely reprocessed and has not exceeded its maximum permissible storage period in a storage cabinet.

In addition to the endoscopes that are currently available, endoscopes that are currently in use or undergoing reprocessing but will be fully reprocessed and thus available by the time a procedure is scheduled to begin may also be taken into account in the availability check. Known durations of procedures and individual reprocessing processes are used here to make the most accurate prediction possible.

Dashboard

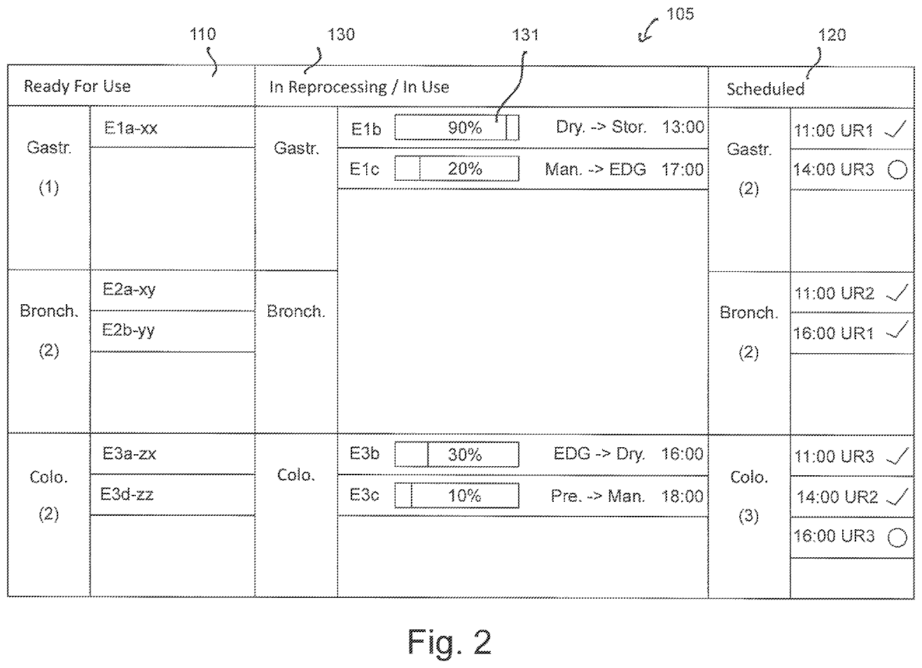

The provisioning system 100 further comprises a user interface 104 through which the determined information about the availability of endoscopes is displayed. A key element of the user interface 104 is a graphical user interface 105 in which the information is intuitively displayed. This user interface 105 is also referred to as a “dashboard”. One possible embodiment of the user interface 105 is shown in FIG. 2.

The user interface 105 has a first presentation area 110 in which data about the endoscopes currently available for use is displayed. A second presentation area 120 displays data about scheduled procedures. A third presentation area 130 displays data about endoscopes currently undergoing reprocessing.

The first presentation area 110, the second presentation area 120, and the third presentation area 130 are arranged along a main direction, in the example shown along a horizontal line. Thereby, the third presentation area 130 can be arranged between the first presentation area and the third presentation area.

In the presentation areas 110, 120,130, data relating to individual endoscopes and/or procedures are arranged along a second main direction, which can be perpendicular to the first main direction. In the example shown, the second main direction is a vertical line.

First Presentation Area

To display the available endoscopes in the first presentation area 110, the type designations and/or serial numbers of the available endoscopes can be listed. The display can be sorted and/or grouped according to endoscope types. Additional grouping may be based on the type of procedure for which the respective endoscopes are suitable. For the groups thus formed, the number of endoscopes available in each group can be output to allow rapid acquisition of the relevant information by an observer.

The sorts and/or groupings to be applied can be configured by the user. Similarly, the user can define what information is to be displayed in the first presentation area. For example, the display of serial numbers of available endoscopes can be enabled or disabled. As further information, a remaining storage time for each available endoscope can be displayed.

In the example shown, the first presentation area 110 indicates that one gastroscope is available, namely gastroscope. E1a with serial number E1a-xx. Furthermore, two bronchoscopes are available, namely bronchoscopes E2a, E2b with serial numbers E2a-xy and E2b-yy. In addition, two colonoscopes are E3a, E3d with serial numbers E3a-zy and E3d-zz.

Second Presentation Area

The second presentation area 120 displays the scheduled procedures. The display can include, for example, the type of procedure, the planned start, and the examination room. Similar to the first presentation area, the second presentation area may be sorted and/or grouped, for example, by type of scheduled procedure. Again, a number of scheduled procedures may be displayed for each group of procedures for quick acquisition of information.

Also, for the second presentation area, a user can configure the type and amount of information displayed. For example, a user can use a filter to control the tune period for which scheduled procedures are displayed and/or select only certain types of procedures for being displayed.

In the example shown, the second presentation area 120 indicates that two gastroscopies are scheduled, at 11:00 h in room UR1 and at 14:00 h in UR3. Furthermore, two bronchoscopies are scheduled, namely at 11:00 h in room UR2 and at 16:00 h in room UR1. Finally, three colonoscopies are scheduled, namely at 11:00 h in room UR3, at 14:00 h in room UR2, and at 16:00 h room UR3.

Third Presentation Area

The third presentation area 130 displays the endoscopes currently undergoing reprocessing. Here, for example, a remaining duration of the reprocessing process and/or the predicted time of availability can be displayed. In addition, the reprocessing step which the endoscope is currently undergoing can be displayed, as well as the remaining duration of the reprocessing step. Furthermore, a visualization 131 in the form of a percentage and/or progress bar can be used to quickly and unambiguously detect the progress of the reprocessing process.

In addition to the endoscopes currently undergoing reprocessing, the third presentation area can also display endoscopes that are currently in use.

Similar to the first and second presentation areas, the endoscopes displayed in the third presentation area can be sorted and/or grouped by endoscope type and/or procedure type. Again, the number of endoscopes in each group can be displayed for quick reference.

In the example shown, gastroscopes E1b and E1c are currently undergoing reprocessing, with reprocessing of gastroscope E1b 90% complete, so gastroscope E1b will be ready for use at 13:00. Gastroscope E1b is currently in the drying process and is awaiting transfer to storage. The reprocessing of gastroscope E1c is 20% complete and it will be ready for use at 17:00. Gastroscope E2c is currently in the manual pre-cleaning process and awaiting transfer to an endoscope reprocessor.

The display of information on the availability of endoscopes in the three presentation areas described above enables a user of the provisioning system to see at a single glance whether the required endoscopes are available or will be available on time for all procedures planned in the selected time period.

For example, it is immediately apparent that gastroscope E1a is ready for the gastroscopy planned at 11:00, and that gastroscope E1b, which is required for the gastroscopy planned at 14:00, will already be fully reprocessed at 13:00.

Bronchoscopes E2a and E2b are ready for use for the bronchoscopies scheduled at 11:00 and 16:00.

For the colonoscopies at 11:00 and 14:00, the E3a and E3d colonoscopes are ready for use. Furthermore, it is immediately apparent that colonoscope E3b will be ready for use at 16:00 and can be used in the colonoscopy scheduled for 16:00.

In order to support rapid visual recognition, the information in ale respective presentation areas can be highlighted in color or provided with pictograms. For example, in the second presentation area 120, those scheduled procedures for which endoscopes are ready for use can be highlighted in green and/or provided with a “tick” symbol, Procedures for which the endoscopes are currently being reprocessed but are to be available in time can be highlighted in yellow and/or provided with a “circle” symbol. Procedures for which an endoscope is not expected to be available in time can be highlighted in red and/or marked with a “triangle” symbol.

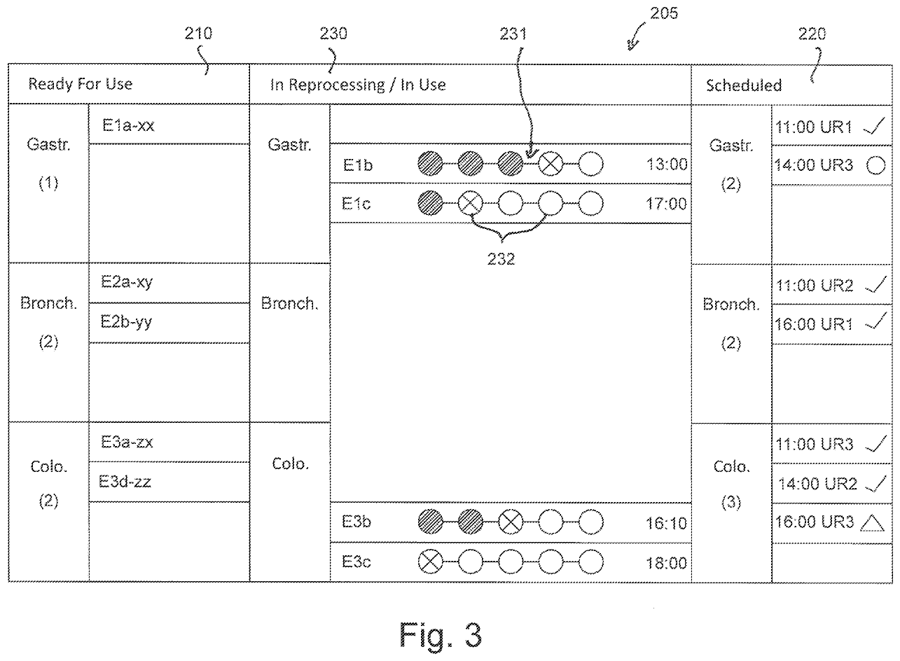

FIG. 3 illustrates another possible embodiment of the user interface 205. It again comprises a first presentation area 210, a second presentation area 220, and a third presentation area 230. Here, the first presentation area 210 and the second presentation area 220 are structured in the same way as the corresponding presentation areas 110, 120 of the user interface 105. The third presentation area 230, however, differs in layout from the third presentation area 130.

In the third presentation area 230, the reprocessing progress of the displayed endoscopes is represented by a visualization 231 in the form of a “string of pearls” with several visualization elements 232, in the example shown as nodes, each node representing a specific step of the reprocessing process. Here, the nodes are represented differently depending on whether the corresponding reprocessing step is still pending, currently being carried out, or already completed. For example, completed steps can be represented with a filled node and pending steps can be represented with a bordered node, while steps currently carried out can be represented with a cross, for example. Other representations are, of course, equally possible.

Similar to the third presentation area 130 of FIG. 2, the third presentation area 230 of FIG. 3 also displays the time at which the corresponding endoscope will be ready for use.

As a further special feature, the display of the endoscopes undergoing reprocessing in the third presentation area 230 is shifted downwards to such an extent that it is displayed below the indication of the endoscopes ready for use in the first presentation area 210. This shift results in the endoscopes undergoing reprocessing in the third presentation area 230 being displayed at the same level as the planned procedures in the second presentation area 220 in which the respective endoscope is to be used, if the displays are sorted accordingly. The interrelated data in the presentation areas 210, 220, 230 are thus aligned in the second main direction, in the example in vertical direction. In this way, a user of the provisioning system 100 can even more easily see whether a suitable endoscope will be available in time for each planned procedure.

In the example shown in FIG. 3, the provisioning of the colonoscope E3b has been delayed by 10 minutes compared to the situation shown in FIG. 2. Such delays can occur, for example, when fluctuations in water pressure or water temperature of the water supply cause a dosing or heating process in an endoscope reprocessor to take longer than planned. Therefore, the colonoscopy scheduled for 16:00 is marked with a “triangle” symbol to indicate that an adjustment to the occupancy schedule may be necessary here.

Additional information can be used to increase the accuracy of the prediction.

Duration and Progress of a Procedure

The average duration of certain standard procedures such as a gastroscopy or colonoscopy is fairly well known. When determining the expected duration of a procedure, additional historical data may be taken into account. For example, the durations of previous examinations of the same patient can be used, as well as examinations of other patients performed by the same physician and/or in the same examination room. This can significantly increase the prediction accuracy for the duration of the examination.

For this purpose, the durations of past procedures are stored in a database implemented in the data processing system 102, in this regard, personal data regarding the patient or physician may be suitably anonymised.

Now, in order to determine when a current procedure will be completed so that the endoscope used can be transferred to reprocessing, the provisioning system can apply various filters to the stored data, and then statistically evaluate the filtered data.

The main filters to be considered here are the type of procedure and the physician performing the procedure. An average value of the procedure duration can then be determined from the data filtered in this way. Alternatively, or additionally, a trend analysis can be performed, by which e.g., a learning curve of a physician is taken into account, during which the duration of the procedures slowly decreases.

However, deviations from the statistically determined duration may occur due to different anatomical conditions and/or complications. To detect such deviations early and determine their impact on endoscope availability planning, progress can be continuously determined during the procedure and transmitted to the provisioning system.

Standard endoscopic procedures are usually divided into predetermined sections, which are defined, for example, by reaching certain anatomical landmarks. Reaching such a landmark is usually documented by storing a still image of the anatomical landmark.

Based is the already stored still images, it is thus possible to estimate how far the procedure has progressed. Deviations from an average procedure progress can thus be detected at an early stage and can be taken into account in availability planning.

Manual Pre-Cleaning

Before reprocessing by machine, endoscopes must be pre-cleaned manually to remove coarse contaminants. The duration of pre-cleaning is mainly dictated by predefined protocols, but can also vary depending on the specialist performing the pre-cleaning and/or the pre-cleaning station used.

As soon as an endoscope is registered at a pre-cleaning station, information is available to the provisioning system as to which specialist is responsible for pre-cleaning. The provisioning system may then use historical data to estimate the duration of the pre-cleaning process. For example, over a longer period of time, it can be determined how much time it took each specialist to pre-clean each type of endoscope. If necessary, more detailed estimates can also be made, which take into account a day of the week or a time of day. Likewise, only those pre-cleaning, processes can be evaluated that were performed at the same pre-cleaning station.

Here, too, the determination is made by filtering and statistically evaluating stored data on past processes.

Placement Within Endoscope Reprocessing Devices

Modern endoscope reprocessing devices are usually designed for reprocessing several endoscopes in one operation. Due to the fact that several endoscopes are reprocessed simultaneously, delays in the provision of individual endoscopes also affect other endoscopes which are to be reprocessed at the same time as the delayed endoscope.

Such transfer of delays can be detected by the provisioning system and taken into account in availability planning.

Progress of Machine Reprocessing

The process duration of machine reprocessing is essentially determined by fixed reprocessing programs. Nevertheless, time deviations may occur.

For example, endoscope reprocessing devices usually perform a leakage test for the endoscopes to be reprocessed. For this purpose, the endoscopes are pressurized with a pressurized gas and the pressure curve is monitored. Since the flow rate of a pump used to build up pressure can change over time, there may be gradual changes in the duration of the leakage test.

Other reasons for variations can be water pressure fluctuations or changes in water temperature that result in altered dosing or heating times. Such variations can be random or systematic. Similarly, gradual changes in the delivery or heating capacity of individual units of an endoscope reprocessing machine can be the cause of changes in process duration.

Newer generations of endoscope reprocessing machines provide information about process progress via a data interface so that this information can be taken into account directly by the provisioning system.

Older endoscope reprocessing machines may not provide such progress information. Nevertheless, a fairly accurate estimate of process progress can be made here, for example, by evaluating historical data on how long a particular reprocessing program usually lasts on a particular endoscope reprocessing machine. Through this, systematic deviations from a usual process duration can also be predicted and taken into account by the provisioning system. The evaluation of historical data is similar to that described with respect to procedure duration or pre-cleaning.

Drying and Storage

After reprocessing by machine, endoscopes are usually transferred to drying cabinets for drying under controlled ambient conditions. The duration of the drying process is hardly subject to fluctuations. After drying, endoscopes are usually transferred to storage cabinets, where they are also stored under controlled environmental conditions until their next use. Here, the maximum permissible storage period is limited; after exceeding the storage period, an endoscope may have to be reprocessed.

Measures in Case of Deviations

If the evaluation of the availability of the endoscopes provides indications that a suitable endoscope will not be available in time for a planned examination, measures may have to be taken to counteract this.

In a simple case like the situation shown in FIG. 3, where an endoscope will be delayed by a few minutes, the planned procedure can be postponed slightly. To do this, it may simply be necessary to inform the patient of the postponement.

However, in unfavorable cases, delaying a procedure can lead to a chain of further delays, which can cause resentment among patients and staff. Therefore, it may be helpful to provide other possible countermeasures against a delay. For this reason, the provisioning system 100 is configured to allow dynamic adjustment of reprocessing processes for endoscopes.

Typically, the steps to be performed during the reprocessing of an endoscope are fixed by protocols. These protocols specify, among other things, how long channels of an endoscope are to be cleaned with a brush during pre-cleaning, which exposure times, active agent concentrations, and process temperatures are to be observed during machine reprocessing, and under which ambient conditions an endoscope is to be dried. The appropriate protocols are usually specified by the endoscope manufacturer to ensure effective reprocessing.

The effectiveness of reprocessing is usually defined by the achieved reduction in the number of colony forming microorganisms (CPU, “Colony Forming Units”). In this regard, each step in a given reprocessing protocol results in a certain reduction in CPU, and the individual reductions add up or multiply to the total reduction achieved (the reduction is most often expressed logarithmically e.g., in a number of powers of ten by which the number of CFU is reduced).

The provisioning system 100 includes a control memory 102a in which rules are stored according to which the process parameters of the predetermined preparation steps can be changed without affecting the effectiveness of the preparation processes. The control memory 102a may be part of the data processing system 102.

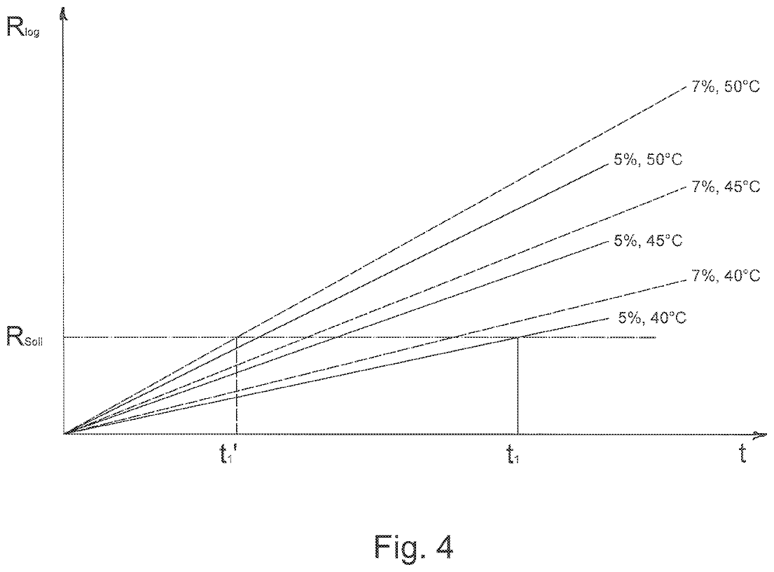

For example, the control memory 102a may store a relationship between individual process parameters of an individual reprocessing step and its effectiveness in the for a of a performance curve field. Such a performance curve field is exemplarily shown in FIG. 4.

FIG. 4 shows the relationship between a reduction in CFU in a disinfection step in an endoscope reprocessing machine. The reduction R is plotted logarithmically as R_{log} on the vertical axis of the characteristic curve. The process time t is plotted linearly on the longitudinal axis.

In the disinfection step, the endoscope is exposed to a disinfection solution of a given concentration for the process time t at a given temperature. The performance curves in FIG. 4 show the course of the reduction of the CPU at different concentrations and temperatures. Solid lines show the time dependent reduction of CPU at a concentration of the disinfectant solution of 5%, and at temperatures of 40° C., 45° C., and 50° C. Dashed lines show the time dependent reduction at a concentration of 7%, also at temperatures of 40° C., 45° C., and 50° C. It can be seen that a given reduction of Rₛₒₗₗ is achieved after a regular process time of t1, when a concentration of 5% and a temperature of 40° C. are set. In contrast, the same reduction Rₛₒₗₗ is achieved after a much shorter process time t1′ if a concentration of 7% and a temperature of 50° C. are set.

The performance curve field shown in FIG. 4 serves only as an example. Similar per curve field can be drawn up for other preparation steps.

In the event of a delay in the provision of a required endoscope, as shown in FIG. 3 for endoscope E3b the provisioning system 100 may be arranged to propose measures to compensate for or reduce the effects of the delay.

For this purpose, the provision system can execute a computer program which examines the performance curves stored in the control memory 102a to determine whether a reprocessing step still to be performed for the endoscope concerned can be modified in such a way that the delay is compensated. Safety limits of individual parameters can be observed in order to avoid damage to the endoscope.

Identified options for adjusting the reprocessing process may be offered to a user of the provisioning system for selection. The user can then decide, e.g., based on further considerations, whether or not to perform a modification. In doing so, the user can weigh economic effects of the modification, such as increased wear and tear on the endoscope, against the effects of the delay, such as disruption of scheduled procedures.

In addition to separate modification of individual reprocessing steps in which the reduction in CPU is maintained for each reprocessing step, successive reprocessing steps can also be modified such that a change in reduction at one step is offset by an opposite change in reduction at another step.

The determination and offering of adjustments can be triggered by the user by activating a button in the graphical user interface. For this purpose, for example, a data element in the second presentation area 120, 220 representing the procedure affected by a delay may be implemented as an interactive button, the activation of which by a graphical input device such as a mouse, or by touch when the user interface is displayed on a touch-sensitive screen, triggers the determination. Determined adjustments can then be displayed in the form of a list, e.g., in a “pop-up” window. The entries of this list may in turn be implemented as interactive buttons, upon activation of which the corresponding customization is implemented.

To implement an adjustment, the provisioning system 100 may send one or more control commands to an affected endoscope reprocessing device, and/or transmit instructions in text form to a manual reprocessing station for display.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. A provisioning system for endoscopes in an application environment comprising one or more examination rooms and one or more reprocessing stations for endoscopes, the provisioning system comprising:
a processor comprising hardware, the processor being configured to:
receive first information about scheduled procedures,
receive second information about a status of one or more endoscopes, the second information comprising an indication of whether an endoscope is ready for use, currently in use, or currently being reprocessed,
determine from the first information and the second information whether a ready-to-use endoscope is or will be available for each respective scheduled procedure, and
control a display to display an indicator which indicates whether a ready-to-use endoscope is or will be available for the respective scheduled procedure.

2. The provisioning system according to claim 1, wherein the processor is further configured to control a display to display a user interface having a graphical user interface, wherein the graphical user interface comprises:
a first presentation area in which data about ready-to-use endoscopes is displayed,
a second presentation area in which data about scheduled procedures is displayed, and
a third presentation area in which data about endoscopes undergoing reprocessing is displayed,
wherein the data displayed in the second presentation area comprises the indicator.

3. The provisioning system according to claim 2, wherein the second information additionally comprises information about a location of the one or more endoscopes.

4. The provisioning system according to claim 1, wherein the processor is further configured, in a case in which an endoscope will not be available in time for a scheduled procedure, to determine one or more modifications to ongoing or pending reprocessing processes by which a delay in the provision of the endoscope can be reduced.

5. The provisioning system according to claim 4, wherein the processer is further configured to determine the one or more modifications upon request by a user.

6. The provisioning system according to claim 4, wherein the processor is configured to control the display to display the determined modifications in a selection list.

7. The provisioning system according to claim 6, wherein the processor is further configured to generate and send control commands to an endoscope reprocessing device upon selection of a determined modification by the user in order to cause the endoscope reprocessing device to perform a modified reprocessing process.

8. The provisioning system according to claim 6, wherein the processor is further configured, upon selection of a determined modification by the user, to generate and transmit execution instructions for a modified reprocessing process to a manual pre-cleaning station.

9. The provisioning system according to claims 4, further comprising a control memory for storing rules according to which process parameters of reprocessing processes can be modified without impairing an effectiveness of the reprocessing processes.

10. The provisioning system according to claim 9, wherein performance curve fields are stored in the control memory, the performance curve fields describing the effectiveness of a reprocessing step as a function of one or more process parameters.

11. A method for operating a provisioning system for endoscopes, comprising:
receiving first information about scheduled procedures,
receiving second information about a location and a status of one or more endoscopes, the second information comprising an indication of whether an endoscope is ready for use, currently in use, or currently being reprocessed;
determining, from the first information and the second information, whether a ready-to-use endoscope is or will be available for each respective scheduled procedure; and
displaying an indicator is displayed in the second presentation area, which indicates whether a ready-to-use endoscope is or will be available for the respective scheduled procedure.

12. The method according to claim 11, wherein the processor is further configured to control the display to display the first and second information in a graphical user interface, wherein the graphical user interface comprises:
a first presentation area in which data about ready-to-use endoscopes is displayed,
a second presentation area in which data about scheduled procedures is displayed, and
a third presentation area in which data about endoscopes undergoing reprocessing is displayed.

13. The method according to claim 12, wherein the second information additionally comprises information about a location of the one or more endoscopes.
